(19) United States
(12) Patent Application Publication
(10) Pub. No.: US 2021/0056041 A1
(43) Pub. Date: Feb. 25, 2021

(54) METHODS AND APPARATUSES FOR STORING ULTRASOUND DATA

(71) Applicant: Butterfly Network, Inc., Guilford, CT (US)

(72) Inventor: Nevada J. Sanchez, Guilford, CT (US)

(73) Assignee: Butterfly Network, Inc., Guilford, CT (US)

(21) Appl. No.: 17/000,142

(22) Filed: Aug. 21, 2020

Related U.S. Application Data
(60) Provisional application No. 62/891,253, filed on Aug. 23, 2019.

(51) Int. Cl. G06F 12/10

(52) U.S. Cl. CPC G06F 12/10; G16H 30/00

(57) ABSTRACT

Aspects of the technology described herein relate to storing ultrasound data. Some embodiments include outputting first ultrasound data from first receive circuitry and outputting second ultrasound data from second receive circuitry on a single clock cycle, and writing the first ultrasound data at a first memory address of a first memory and writing the second ultrasound data at a second memory address of a second memory, where the first and second memory addresses are different. Some embodiments include outputting ultrasound data and a memory address, remapping the memory address to generate a remapped memory address, and writing the ultrasound data to memory at the remapped memory address.

### CROSS-REFERENCE TO RELATED APPLICATIONS

This application claims the benefit under 35 U.S.C. § 119(e) of U.S. Patent Application Ser. No. 62/891,253, filed Aug. 23, 2019 under Attorney Docket No. B1348.70151US00, and entitled “METHODS AND APPARATUSES FOR STORING ULTRASOUND DATA,” which is hereby incorporated by reference herein in its entirety.

### FIELD

Generally, the aspects of the technology described herein relate to storing ultrasound data. Certain aspects relate to remapping memory addresses and/or storing different ultrasound data received at the same time at different memory addresses of different memories.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an ultrasound apparatus comprises first receive circuitry, second receive circuitry, first memory, and second memory. The ultrasound apparatus is configured to output first ultrasound data from the first receive circuitry and output second ultrasound data from the second receive circuitry on a single clock cycle, and write the first ultrasound data at a first memory address of the first memory and write the second ultrasound data at a second memory address of the second memory. The first and second memory addresses are different.

In some embodiments, the ultrasound apparatus further comprises memory address circuitry configured to generate the first memory address and the second memory address. In some embodiments, the memory address circuitry is configured to remap a memory address received from the first receive circuitry to generate the first memory address and to remap a memory address received from the second receive circuitry to generate the second memory address. In some embodiments, the memory address circuitry is configured to add a memory address received from the first receive circuitry to a first seed value in order to generate the first memory address, and add a memory address received from the second receive circuitry to a second seed value in order to generate the second memory address. The first seed value and the second seed value are different. In some embodiments, the memory address circuitry is configured to add a memory address received from the first receive circuitry to a first seed value in order to generate a first sum, add a memory address received from the second receive circuitry to a second seed value in order to generate a second sum, gray encode the first sum in order to generate the first memory address, and gray encode the second sum in order to generate the second memory address. The first seed value and the second seed value are different. In some embodiments, the memory address circuitry is configured to add a memory address received from the first receive circuitry to a first seed value in order to generate a first sum, add a memory address received from the second receive circuitry to a second seed value in order to generate a second sum, generate a first pseudorandom value based on the first sum, wherein the first pseudorandom value is the first memory address, and generate a second pseudorandom value based on the second sum, wherein the second pseudorandom value is the first memory address. The first seed value and the second seed value are different.

In some embodiments, the memory address circuitry is configured to generate a counter value on each clock cycle, add the counter value to a first seed value in order to generate the first memory address, and add the counter value to a second seed value in order to generate the second memory address. The first seed value and the second seed value are different. In some embodiments, the memory address circuitry is configured to generate a counter value on each clock cycle, add the counter value to a first seed value in order to generate a first sum, add the counter value to a second seed value in order to generate a second sum, gray encode the first sum in order to generate the first memory address, and gray encode the second sum in order to generate the second memory address. The first seed value and the second seed value are different. In some embodiments, the memory address circuitry is configured to generate a counter value on each clock cycle, add the counter value to a first seed value in order to generate a first sum, add the counter value to a second seed value in order to generate a second sum, generate a first pseudorandom value based on the first sum, wherein the first pseudorandom value is the first memory address, and generate a second pseudorandom value based on the second sum, wherein the second pseudorandom value is the first memory address. The first seed value and the second seed value are different.

In some embodiments, the memory address is configured to generate a first pseudorandom value based on a first seed value, wherein the first pseudorandom value is the first memory address, and generate a second pseudorandom value based on a second seed value, wherein the second pseudorandom value is the second memory address. The first seed value and the second seed values are different. In some embodiments, the ultrasound apparatus further comprises pseudorandom value generation circuitry configured to generate the first and second pseudorandom values. In some embodiments, the pseudorandom value generation circuitry comprises a linear-feedback shift register (LFSR).

In some embodiments, the ultrasound apparatus further comprises storage circuitry for storing the first and second seed values. In some embodiments, the first seed value is related to a location of the first receive circuitry and the second seed value is related to a location of the second receive circuitry. In some embodiments, the location of the first receive circuitry and the location of the second receive circuitry are locations in an ultrasound-on-chip. In some embodiments, the ultrasound apparatus further comprises pseudorandom value generation circuitry for generating the first and second seed values. In some embodiments, the pseudorandom value generation circuitry comprises a linear-feedback shift register (LFSR).

In some embodiments, the memory address received from the first receive circuitry and the memory address received from the second receive circuitry are the same. In some embodiments, the first receive circuitry comprises a first counter, the address received from the first receive circuitry is generated by the first counter, the second receive circuitry comprises a second counter, and the address received from the second receive circuitry is generated by the second counter. In some embodiments, the first receive circuitry comprises first circuitry configured to generate addresses not in succession, the address received from the first receive circuitry is generated by the first circuitry, the second receive circuitry comprises second circuitry configured to generate addresses not in succession, and the address received from the second receive circuitry is generated by the second circuitry.

In some embodiments, the first and second circuitry comprises beamforming circuitry. In some embodiments, the ultrasound apparatus is configured, when writing the first ultrasound data at the first memory address of the first memory and writing the second ultrasound data at the second memory address of the second memory, to sum the first ultrasound data with existing data at the first memory address of the first memory, and sum the second ultrasound data with existing data at the second memory address of the second memory. In some embodiments, the ultrasound apparatus is configured, when writing the first ultrasound data at the first memory address of the first memory and writing the second ultrasound data at the second memory address of the second memory, to overwrite existing data at the first memory address of the first memory with the first ultrasound data, and overwrite existing data at the second memory address of the second memory with the second ultrasound data. In some embodiments, the first and second receive circuitry each comprise amplification circuitry, analog filtering circuitry, analog beamforming circuitry, analog dechirp circuitry, analog quadrature demodulation (AQDM) circuitry, analog time delay circuitry, analog phase shifter circuitry, analog summing circuitry, analog time gain compensation circuitry, analog averaging circuitry, analog-to-digital conversion circuitry, digital filtering, digital beamforming circuitry, digital quadrature demodulation (DQDM) circuitry, digital averaging circuitry, digital dechirp circuitry, digital time delay circuitry, digital phase shifter circuitry, digital summing circuitry, and/or digital multiplying circuitry.

According to another aspect, an ultrasound apparatus comprises receive circuitry, memory, and memory address circuitry. The ultrasound apparatus is configured to output, from the receive circuitry, ultrasound data and a memory address; remap, with the memory address circuitry, the memory address to generate a remapped memory address; and write the ultrasound data to the memory at the remapped memory address.

In some embodiments, the memory address circuitry is configured to add the memory address to a seed value in order to generate the remapped memory address. In some embodiments, the memory address circuitry is configured to add the memory address to a seed value in order to generate a sum and gray encode the sum in order to generate the remapped memory address. In some embodiments, the memory address circuitry is configured to add the memory address to a seed value in order to generate a sum and generate a pseudorandom value based on the sum to generate the remapped memory address.

In some embodiments, the ultrasound apparatus further comprises storage circuitry for storing the seed value. In some embodiments, the seed value is related to a location of the receive circuitry. In some embodiments, the location of the receive circuitry is a location in an ultrasound-on-chip. In some embodiments, the ultrasound apparatus further comprises pseudorandom value generation circuitry for generating the seed value. In some embodiments, the pseudorandom value generation circuitry comprises a linear-feedback shift register (LFSR). In some embodiments, the memory address circuitry is configured to gray encode the memory address in order to generate the remapped memory address.

In some embodiments, the receive circuitry comprises a counter, and the memory address is generated by the counter. In some embodiments, the receive circuitry comprises circuitry configured to generate addresses not in succession, and the memory address is generated by the circuitry. In some embodiments, the circuitry comprises beamforming circuitry.

In some embodiments, writing the ultrasound data to the memory at the remapped memory address comprises summing the ultrasound data with existing data at the remapped memory address of the memory. In some embodiments, writing the ultrasound data to the memory at the remapped memory address comprises overwriting existing data at the remapped memory address of the memory with the ultrasound data. In some embodiments, the receive circuitry comprises amplification circuitry, analog filtering circuitry, analog beamforming circuitry, analog dechirp circuitry, analog quadrature demodulation (AQDM) circuitry, analog time delay circuitry, analog phase shifter circuitry, analog summing circuitry, analog time gain compensation circuitry, analog averaging circuitry, analog-to-digital conversion circuitry, digital filtering, digital beamforming circuitry, digital quadrature demodulation (DQDM) circuitry, digital averaging circuitry, digital dechirp circuitry, digital time delay circuitry, digital phase shifter circuitry, digital summing circuitry, and/or digital multiplying circuitry.

Some aspects include a method to perform the actions that the apparatus is configured to perform.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1A is a schematic diagram illustrating example circuitry in an ultrasound device, in accordance with certain embodiments described herein;

FIG. 1B is a schematic diagram illustrating example circuitry in an ultrasound device, in accordance with certain embodiments described herein;

FIG. 2 is a schematic diagram illustrating another example of circuitry in an ultrasound device, in accordance with certain embodiments described herein;

FIG. 3A is a schematic diagram illustrating another example of circuitry in an ultrasound device, in accordance with certain embodiments described herein;

FIG. 3B is a schematic diagram illustrating another example of circuitry in an ultrasound device, in accordance with certain embodiments described herein;

FIG. 4A is a schematic diagram illustrating another example of circuitry in an ultrasound device, in accordance with certain embodiments described herein;

FIG. 4B is a schematic diagram illustrating another example of circuitry in an ultrasound device, in accordance with certain embodiments described herein;

FIG. 4C is a schematic diagram illustrating another example of circuitry in an ultrasound device, in accordance with certain embodiments described herein;

FIG. 4D is a schematic diagram illustrating another example of circuitry in an ultrasound device, in accordance with certain embodiments described herein;

FIG. 4E is a schematic diagram illustrating another example of circuitry in an ultrasound device, in accordance with certain embodiments described herein;

FIG. 5 is a flow diagram illustrating an example process for storing ultrasound data, in accordance with certain embodiments described herein;

FIG. 6 is a flow diagram illustrating another example process for storing ultrasound data, in accordance with certain embodiments described herein;

FIG. 7 is a block diagram illustrating an example of a downstream portion of the circuitry of FIGS. 1-4E, in accordance with certain embodiments described herein;

FIG. 8 is a block diagram illustrating another example of a downstream portion of the circuitry of FIGS. 1-4E, in accordance with certain embodiments described herein;

FIG. 9 is a perspective view of an example handheld ultrasound probe in which an ultrasound-on-device may be disposed, in accordance with certain embodiments described herein;

FIG. 10 illustrates a subject wearing an example ultrasound patch in which an ultrasound-on-device may be disposed, in accordance with certain embodiments described herein; and

FIG. 11 is a perspective view of an example ultrasound pill in which an ultrasound-on-device may be disposed, in accordance with certain embodiments described herein.

### DETAILED DESCRIPTION

Recent advances in ultrasound technology have enabled large arrays of ultrasound transducers and ultrasound processing units (UPUs) to be incorporated onto an integrated circuit to form an ultrasound-on-chip. Each UPU may include, for example, high-voltage pulsers to drive the ultrasonic transducers to emit ultrasound waves; analog and mixed-signal receiver channels to receive and digitize ultrasound echoes; digital processing circuitry to filter, compress, and/or beamform the digital data from each channel; and digital sequencing circuitry to control and synchronize different parts of the UPU circuitry. An ultrasound-on-chip can form the core of a handheld ultrasound probe or an ultrasound device having another form factor. For further description of ultrasound-on-chips, see U.S. patent application Ser. No. 15/826,711 titled “UNIVERSAL ULTRASOUND IMAGING DEVICE AND RELATED APPARATUS AND METHODS,” filed on Jun. 19, 2017 and published as U.S. Pat. App. Publication No. 2017-0360399 A1 (and assigned to the assignee of the instant application), which is incorporated by reference herein in its entirety.

In some embodiments, the ultrasound-on-chip may include multiple blocks of memory, each block configured to store ultrasound data from a different block of receive circuitry (e.g., circuitry configured to receive and process ultrasound data from different ultrasonic transducers). There may be, for example, on the order of tens, hundreds, or thousands (e.g., 32-1024) blocks of memory. The inventors have recognized that when all the blocks of memory store data at one memory address on one clock cycle and then store data at another memory address on the subsequent clock cycle, in some cases the digital switching activity across all the blocks of memory in switching between certain addresses may cause a draw in current from the power supply, power supply noise, and/or transfer of digital switching activity through capacitive coupling to nearby low bandwidth and/or low amplitude analog signals. This can, in turn, cause noise in images and measurements generated based on the analog signals. In some embodiments, the power disturbances may occur due to switching between two address that have a larger number of bits that flip (i.e., change from 1 to 0 or vice versa) and/or may occur due to switching between two addresses in which higher order (i.e., more significant) bits flip, as the circuitry in the memory may consume more power to flip higher order bits.

The inventors have recognized that such power disturbances may be reduced by implementing memory address circuitry. The memory address circuitry may be configured to remap memory addresses, where remapping a memory address may include mapping a memory address to a new address using a mapping of the memory address space onto itself. In some embodiments, if multiple blocks of receive circuitry output the same memory address for storing ultrasound data on a given clock cycle, the memory address circuitry may be configured to map that memory address to new memory addresses, a different address for each block of receive circuitry. In some embodiments, the memory address circuitry may be configured to generate a different address for each block of receive circuitry on a given clock cycle (without mapping). Thus, each block of receive circuitry (or at least certain blocks of receive circuitry) may write ultrasound data to a different memory address on a given clock cycle. Accordingly, rather than all the blocks of memory simultaneously undergoing a transition from one memory address to another that may cause a power disturbance (e.g., a transition that includes flipping a large number of bits and/or flipping higher order bits), different blocks of memory may undergo these transitions at different times. This may reduce the total power disturbance caused by such transitions at any given time.

In some embodiments, the memory address circuitry may map an address to a new address f(address+seed mod N), where seed is different for each block of receive circuitry, f is a function, and the available memory addresses range from 0 to N−1. In some embodiments, f(address+seed mod N)=(address+seed mod N). In other words, the memory address circuitry may map an address to a different address for each block of receive circuitry, where the different addresses are linearly offset from each other. In some embodiments, f may be a function that transforms a memory address from standard binary coding to gray coding. As another example, f may be a function that transforms a memory address to a pseudorandom memory address. However, these examples are non-limiting, and other schemes and functions f may be used for remapping or generating memory addresses. The seed for a given block of receive circuitry may be, for example, related to the receive circuitry's physical location (e.g., its location in an ultrasound-on-chip) or a pseudorandom value, although other schemes for assigning different seeds to different blocks of receive circuitry may be used. In some embodiments, new addresses may be generated based just on a seed, not an address. In some embodiments, new addresses may be generated based just on an address, not a seed.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

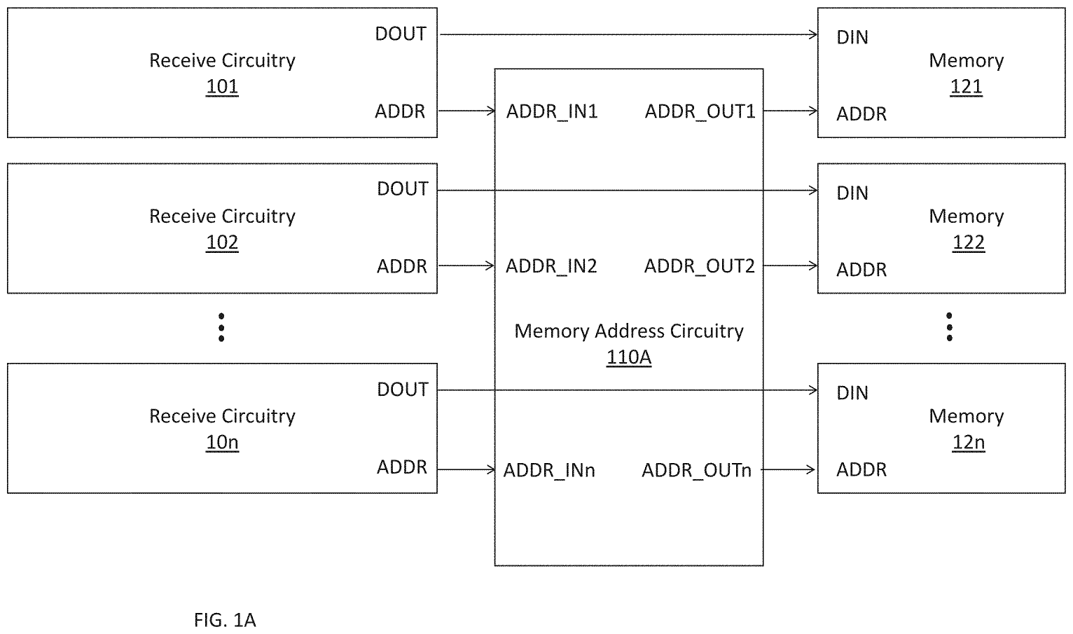

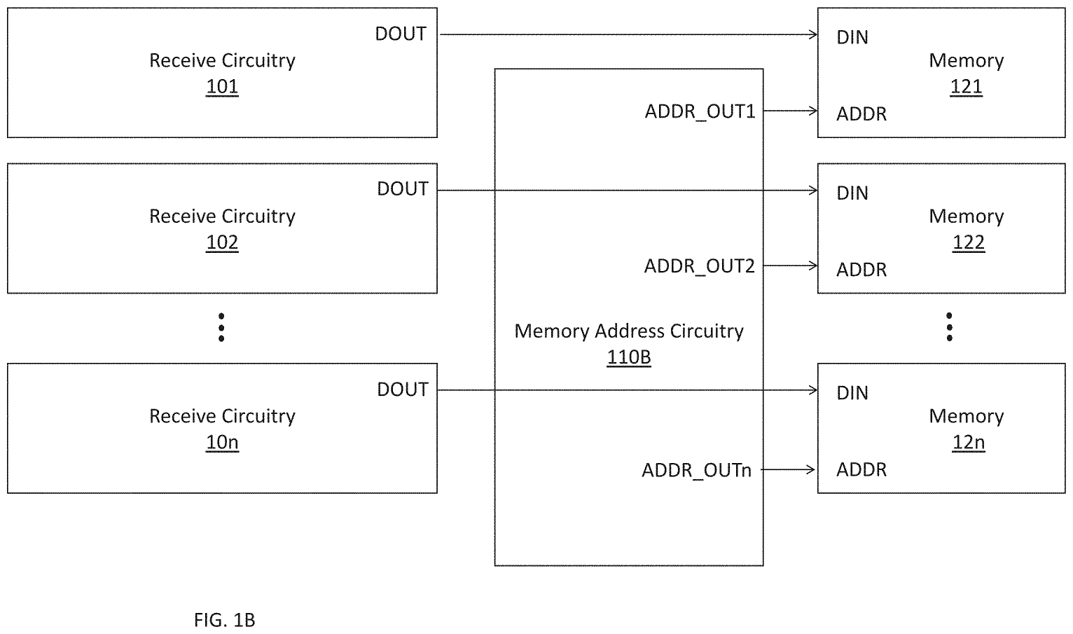

FIG. 1A is a schematic diagram illustrating example circuitry in an ultrasound device, in accordance with certain embodiments described herein. The ultrasound device may be, for example, an ultrasound-on-chip. FIG. 1A includes receive circuitry 101, 102 . . . 10n; memory address circuitry 110; and memory 121, 122 . . . 12n.

Each block of receive circuitry 10i (where i may range from 1 to n) may be configured to generate a word of ultrasound data by receiving one or more ultrasound signals from one or more ultrasonic transducers and processing them. The receive circuitry 10i may include, for example, amplification circuitry, analog filtering circuitry, analog beamforming circuitry, analog dechirp circuitry, analog quadrature demodulation (AQDM) circuitry, analog time delay circuitry, analog phase shifter circuitry, analog summing circuitry, analog time gain compensation circuitry, analog averaging circuitry, analog-to-digital conversion circuitry, digital filtering, digital beamforming circuitry, digital quadrature demodulation (DQDM) circuitry, digital averaging circuitry, digital dechirp circuitry, digital time delay circuitry, digital phase shifter circuitry, digital summing circuitry, and/or digital multiplying circuitry. Each block of receive circuitry 10i includes a data (DOUT) and address (ADDR) output terminal. In operation, each block of receive circuitry 10i may be configured, on a given clock cycle, to output a word of ultrasound data at the DOUT terminal and a memory address at the ADDR terminal for writing the ultrasound data. In some embodiments, to generate a memory address, each block of receive circuitry may include a counter configured to output, at each clock cycle, a value that increases linearly from the previous value (e.g., is the value from the previous clock cycle incremented by 1). However, in some embodiments, the receive circuitry may include circuitry (e.g., beamforming circuitry) configured to output specific addresses that may not be in succession. In some embodiments, each block of receive circuitry 10i may be configured to output the same address on a given clock cycle.

The memory address circuitry 110A includes an address (ADDR_INi) input terminal and an address (ADDR_OUTi) output terminal for each block of receive circuitry 10i. Each ADDR_INi terminal is coupled to the ADDR terminal of the receive circuitry 10i. The memory address circuitry 110A may be configured to receive the memory address from the ADDR terminal of a block of the receive circuitry 10i at the ADDR_INi terminal and output a remapped memory address (i.e., a new address that has been remapped based on the address received from the receive circuitry 10i) at the ADDR_OUTi terminal. In some embodiments, even if each block of receive circuitry 10i outputs the same address at the ADDR terminal on a given clock cycle, the memory address circuitry 110A may be configured to output a different address at each ADDR_OUTi terminal. Further description of the memory address circuitry 110A may be found below.

Each block of memory circuitry 12i includes a data (DIN) input terminal and an address (ADDR) input terminal. The DOUT terminal of each block of receive circuitry 10i is coupled to the DIN terminal of the memory 12i. Each ADDR_OUTi terminal of the memory address circuitry 110A is coupled to the ADDR terminal of the memory 12i. The memory 12i may be configured to write the ultrasound data received at the DIN terminal from the DOUT terminal of the receive circuitry 10i at the address received at the ADDR terminal from the ADDR_OUTi terminal of the memory address circuitry 110A. Writing data to a particular address in memory may include summing the data with the existing data at that address in memory (in other words, accumulating) or overwriting the existing data at that address in memory with the new data.

FIG. 1B is a schematic diagram illustrating example circuitry in an ultrasound device, in accordance with certain embodiments described herein. FIG. 1B illustrates memory address circuitry 110B, which differs from the memory address circuitry 110A in that the memory address circuitry 110B does not receive addresses from receive circuitry 10i as an input. Instead, the memory address circuitry 110B may be configured to internally generate different memory addresses for each block of receive circuitry 10i that are not based on memory addresses received from the receive circuitry 10i. Further description of the memory address circuitry 110B may be found below.

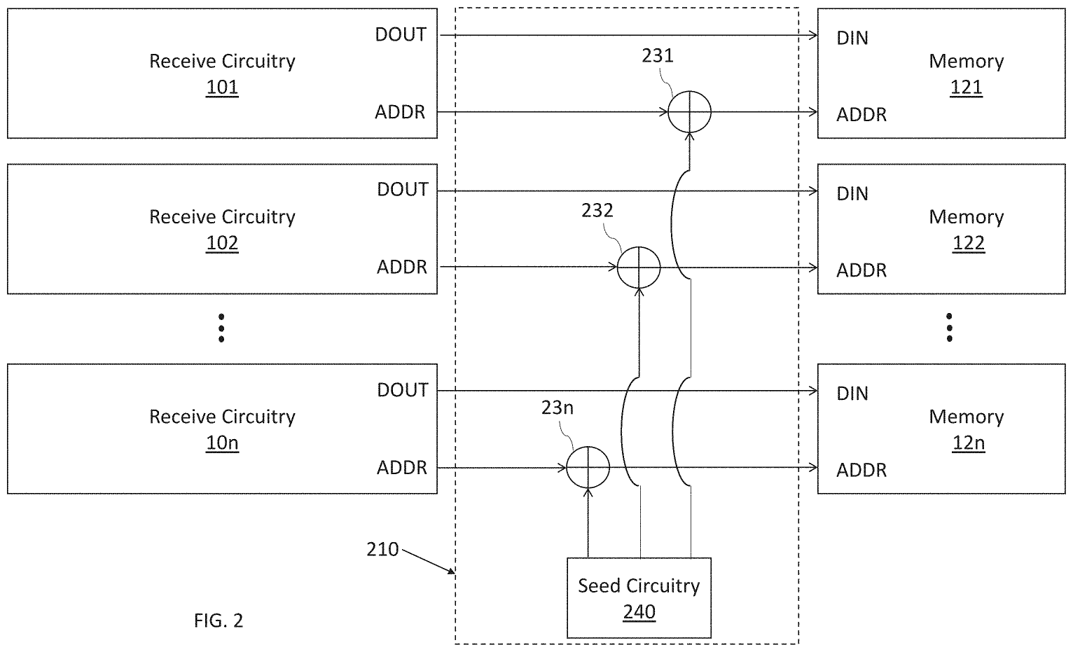

FIG. 2 is a schematic diagram illustrating example circuitry in an ultrasound device, in accordance with certain embodiments described herein. FIG. 2 illustrates detail of memory address circuitry 210, which may be the same as the memory address circuitry 110A in FIG. 1A. The memory address circuitry 210 includes adders 231, 232 . . . 23n and seed circuitry 240. One input of each of the adders 23i is coupled to the ADDR terminal of the receive circuitry 10i, another input is coupled to the seed circuitry 240, and the output is coupled to the ADDR terminal of the memory 12i. In operation, each of the adders 23i may be configured to add the address received from the ADDR terminal of the receive circuitry 10i to a seed value received from the seed circuitry 240. The seed value provided to each adder 23i may be different, and may function as an offset value. Each adder 23i may be configured to provide the sum of the address received from the ADDR terminal of the receive circuitry 10i and the seed value received from the seed circuitry 240 to the ADDR terminal of the memory 12i. The memory 12i may be configured to write the word of ultrasound data received at the DIN terminal from the DOUT terminal of the receive circuitry 10i to the memory address that is equivalent to the sum received from the adder 23i. Writing data to a particular address in memory may include summing the data with the existing data at that address in memory (in other words, accumulating) or overwriting the existing data at that address in memory with the new data. The output of the memory address circuitry 210 thus may be (address+seed mod N), where address is received from the ADDR terminal of the receive circuitry 10i and seed is the particular seed value received from the seed circuitry 240 for the receive circuitry 10i. The modulus N may occur due to the bit widths of the address lines accommodating values from 0 to N−1. Table 1 illustrates examples of addresses, seeds, and remapped addresses, where the remapped address is (address+seed mod N).

TABLE 1
Examples of addresses, seeds, and remapped addresses,
where the remapped address is (address + seed mod N)

| | | | | Remapped | Remapped |
|---|---|---|---|---|---|
| Address | Address | Seed | Seed | Address | Address |
| (Binary) | (Decimal) | (Binary) | (Decimal) | (Binary) | (Decimal) |
| 0111 | 7 | 0001 | 1 | 1000 | 8 |
| 0111 | 7 | 0011 | 3 | 1010 | 10 |
| 1000 | 8 | 0001 | 1 | 1001 | 9 |
| 1000 | 8 | 0011 | 3 | 1011 | 11 |

The seed circuitry 240 may include storage circuitry (e.g., registers) for storing the seed values for each block of receive circuitry. Any scheme may be used by the seed circuitry 240 to assign different seed values (which may function as offset values) to different blocks of receive circuitry 10i. In some embodiments, the seed may be related to the receive circuitry's physical location (e.g., its location in an ultrasound-on-chip). For example, the seed circuitry 240 may provide a seed of 0 to the top block of receive circuitry in the ultrasound-on-chip, a seed of 1 for the next block of receive circuitry, a seed of 2 for the next block of receive circuitry, etc. In some embodiments, the seed may be a pseudorandom value. For example, the seed circuitry 240 may include a linear-feedback shift register (LFSR) configured to generate a different pseudorandom value as the seed for each block of receive circuitry 10i. In some embodiments, the seeds outputted by the seed circuitry 240 may be programmable. For example, the seeds outputted by the seed circuitry 240 may be programmed to change between acquisitions or frames.

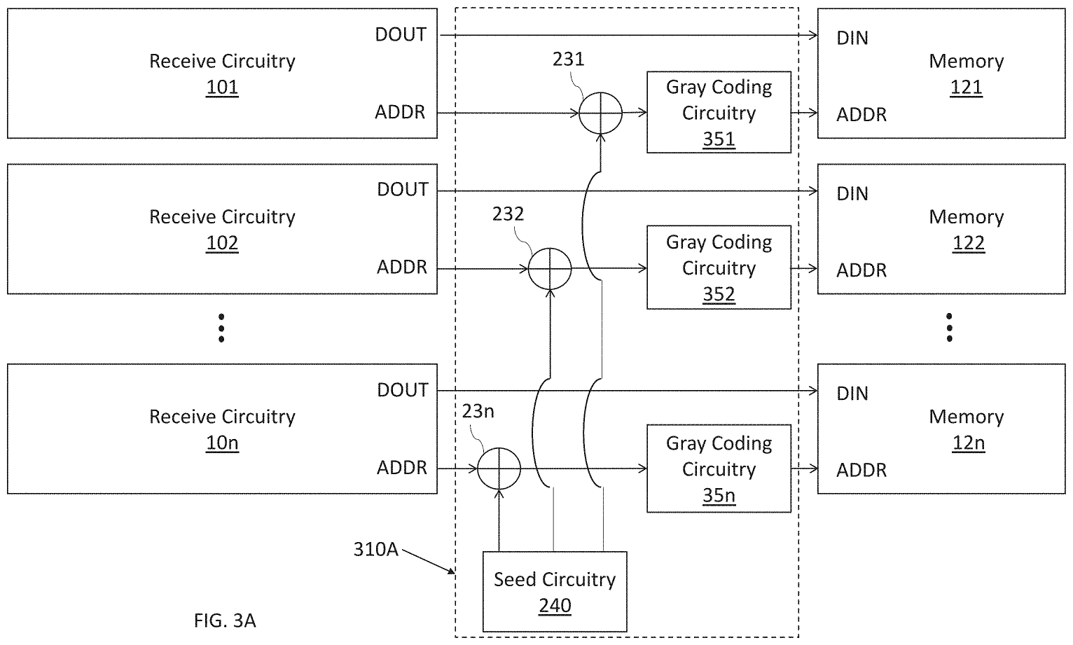

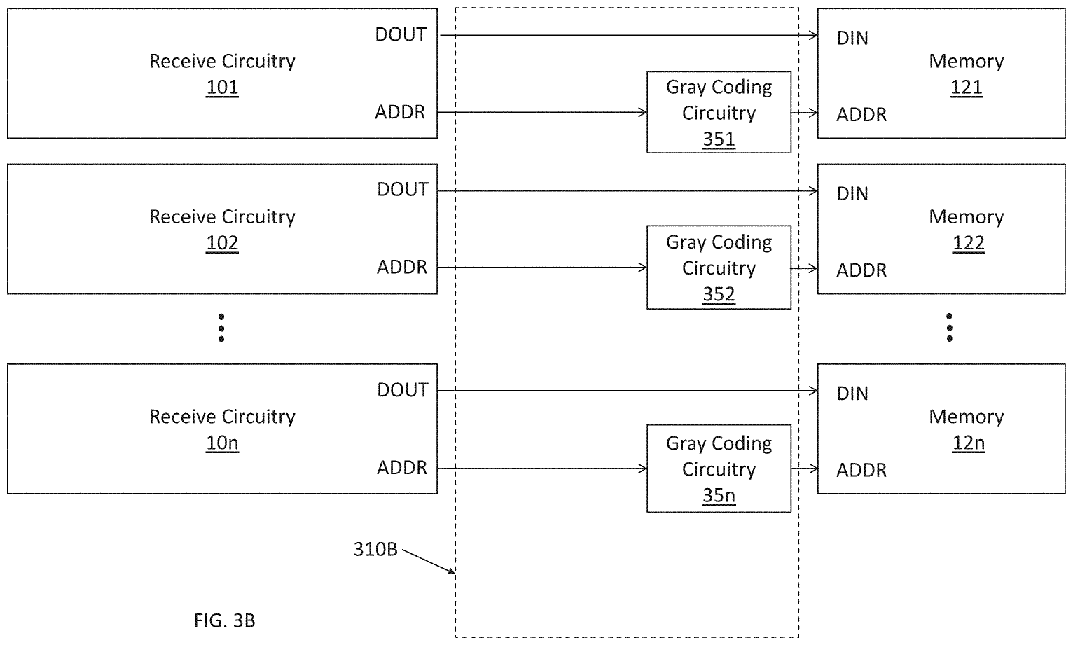

FIG. 3A is a schematic diagram illustrating example circuitry in an ultrasound device, in accordance with certain embodiments described herein. FIG. 3A illustrates detail of memory address circuitry 310A, which may be the same as the memory address circuitry 110A in FIG. 1A. The memory address circuitry 310A differs from the memory address circuitry 210 in that the output of each of the adders 23i is coupled to an input of a block of gray coding circuitry 35i, and the output of each block of gray coding circuitry 35i is coupled to the ADDR terminal of the memory 12i. In operation, each of the adders 23i may be configured to add the address received from the ADDR terminal of the receive circuitry 10i to a seed received from the seed circuitry 240. The seed provided to each adder 23i may be different and may function as an offset value. Each adder 23i may be configured to provide the sum of the address received from the ADDR terminal of the receive circuitry 10i and the seed value received from the seed circuitry 240 to the gray coding circuitry 35i. The gray coding circuitry 35i may be configured to convert the received sum from binary coding to gray coding and provide the gray-coded sum to the ADDR terminal of the memory 12i. The memory 12i may be configured to write the word of ultrasound data received at the DIN terminal from the DOUT terminal of the receive circuitry 10i to the memory address that is equivalent to the gray-coded sum received from the gray coding circuitry 35i. The output of the memory address circuitry 310A thus may be gray_encoding(address+seed mod N), where address is received from the ADDR terminal of the receive circuitry 10i, seed is the seed value received from the seed circuitry 240, and gray_encoding(n) is a function that transforms a binary-coded value n to a gray-coded value. The modulus N may occur due to the bit widths of the address lines accommodating values from 0 to N−1. Table 2 illustrates examples of addresses, seeds, and remapped addresses, where the remapped address is gray_encoding(address+seed mod N).

TABLE 2
Examples of addresses, seeds, and remapped
addresses, where the remapped address is
gray_encoding(address + seed mod N)

| | | | | Remapped | Remapped |
|---|---|---|---|---|---|
| Address | Address | Seed | Seed | Address | Address |
| (Binary) | (Decimal) | (Binary) | (Decimal) | (Binary) | (Decimal) |
| 0111 | 7 | 0001 | 1 | 1100 | 12 |
| 0111 | 7 | 0011 | 3 | 1111 | 15 |
| 1000 | 8 | 0001 | 1 | 1101 | 13 |
| 1000 | 8 | 0011 | 3 | 1110 | 14 |

It should be appreciated that if the sequence of addresses inputted to the memory address circuitry 210 or 310A by the receive circuitry 10i follows linear ordering (e.g., uses standard binary coding), then the sequence of addresses outputted by the memory address circuitry 210 may also follow linear ordering, but the sequence of addresses outputted by the memory address circuitry 310A may follow gray code ordering. If the larger source of power disturbance is digital switching of memory addresses that includes flipping higher order bits, then linearly ordered addresses may reduce power disturbances more than gray-code ordered addresses, because linearly ordered addresses may flip higher order bits less often than gray coded addresses. If the larger source of power disturbance is digital switching of memory addresses that includes flipping large numbers of bits, then gray-code ordered addresses may reduce power disturbances more than linearly ordered addresses, because gray-code ordered addresses may flip only one bit per transition.

FIG. 3B is a schematic diagram illustrating example circuitry in an ultrasound device, in accordance with certain embodiments described herein. FIG. 3B illustrates detail of memory address circuitry 310B, which may be the same as the memory address circuitry 110A in FIG. 1A. The memory address circuitry 310B differs from the memory address circuitry 310A in that the memory address from the ADDR terminal of each block of receive circuitry 10i is coupled to gray coding circuitry 35i, without adders 23i. The output of the memory address circuitry 310B thus may be gray_encoding(address mod N), where address is received from the ADDR terminal of the receive circuitry 10i and gray_encoding(n) is a function that transforms a binary-coded value n to a gray-coded value. The modulus N may occur due to the bit widths of the address lines accommodating values from 0 to N−1. Table 3 illustrates examples of addresses and remapped addresses, where the remapped address is gray_encoding(address mod N).

TABLE 3
Examples of addresses and remapped addresses, where
the remapped address is gray_encoding(address mod N)

| | | Remapped | Remapped |
|---|---|---|---|
| Address | Address | Address | Address |
| (Binary) | (Decimal) | (Binary) | (Decimal) |
| 0111 | 7 | 0100 | 4 |
| 1000 | 8 | 1100 | 12 |

It should be appreciated that in operation, if each block of receive circuitry outputs the same memory address on a given clock cycle, each block of memory 12i may store ultrasound data at the same address on a given clock cycle. However, if the larger source of power disturbance is digital switching of memory addresses that includes flipping large numbers of bits, then it may be sufficient for the memory 12i to use the same gray-coded address on a given clock cycle. Because gray-code ordered addresses flip only one bit per transition. using gray-code ordered addresses may reduce power disturbances to an acceptable degree.

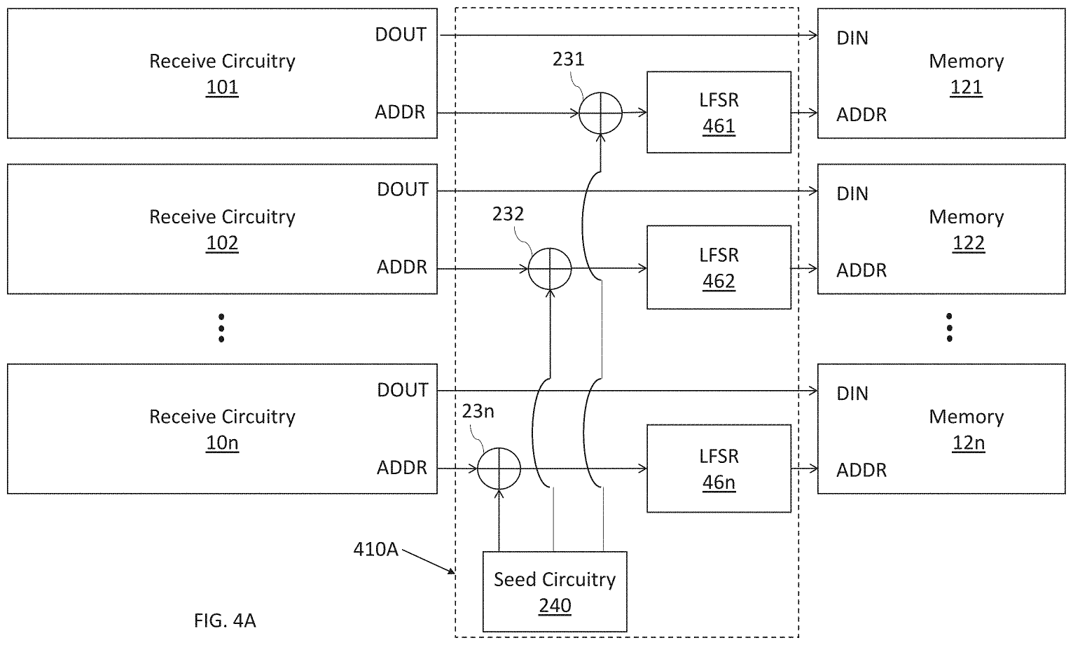

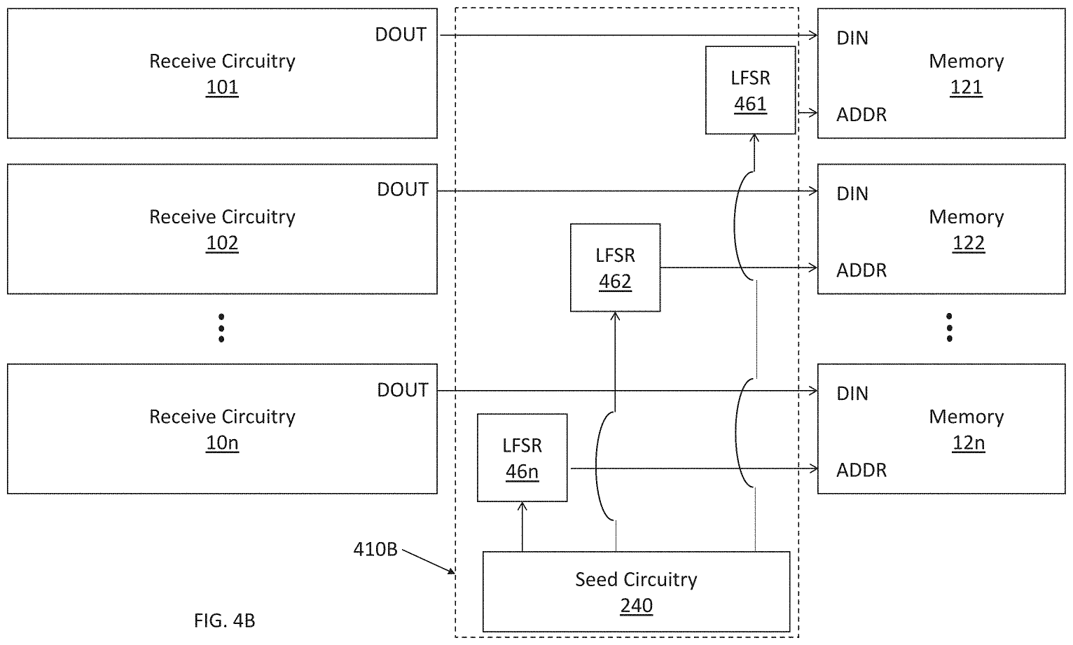

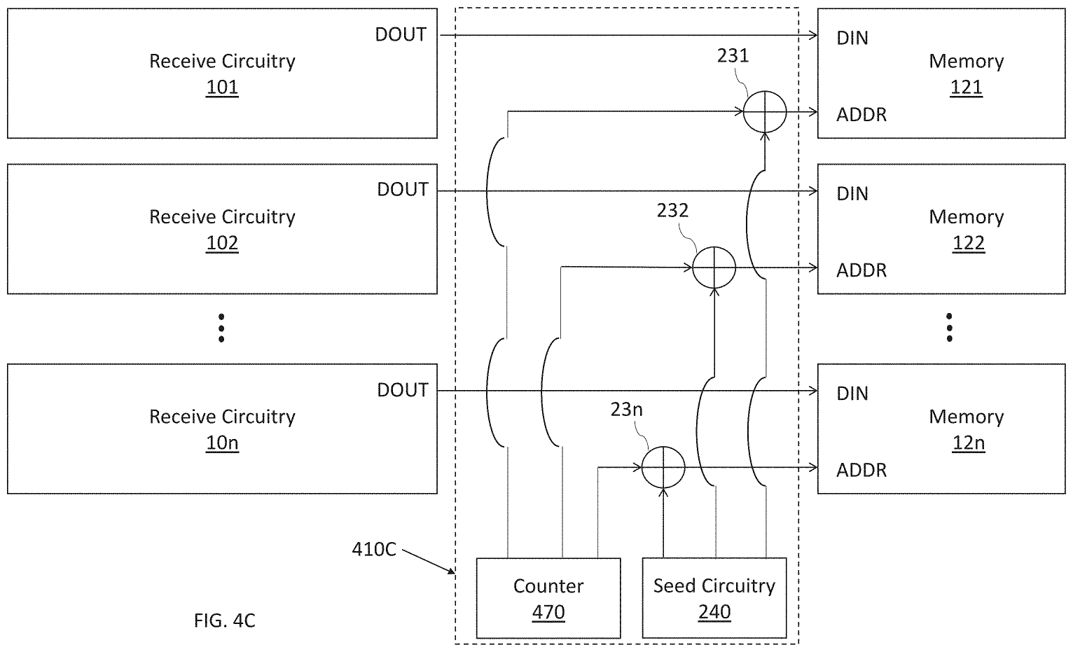

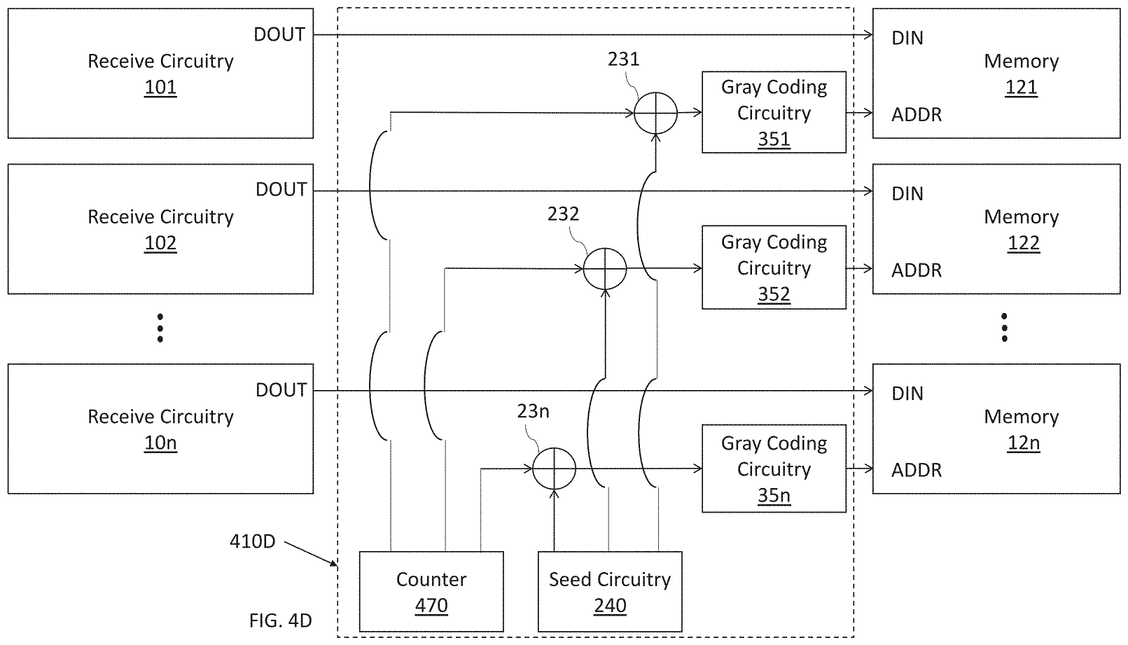

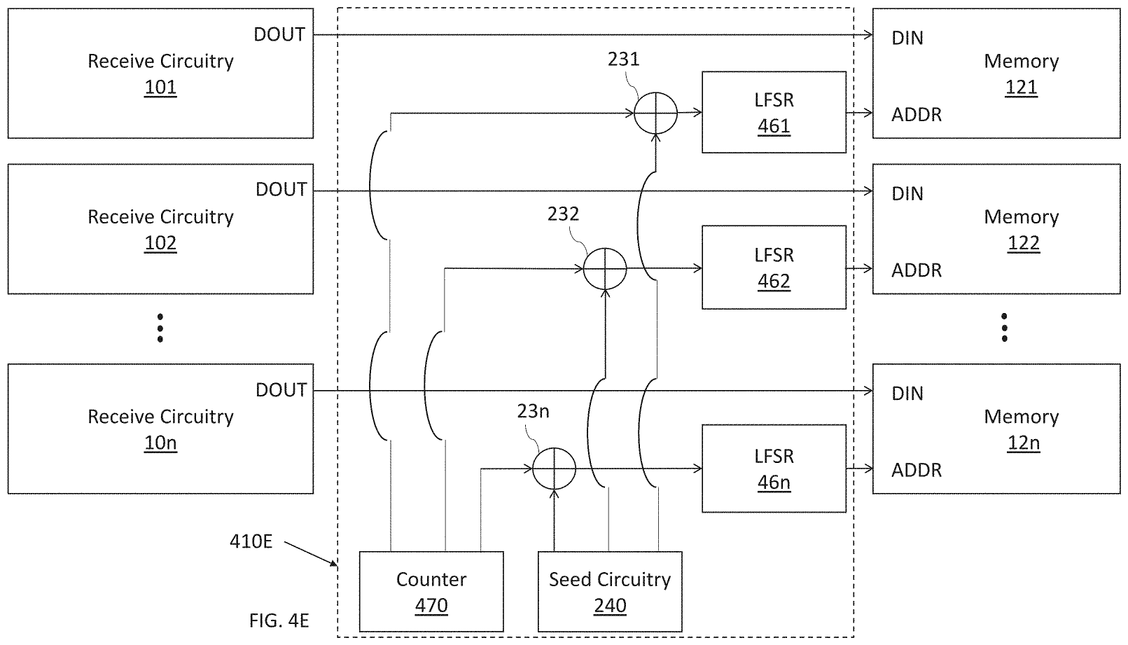

FIG. 4A is a schematic diagram illustrating example circuitry in an ultrasound device, in accordance with certain embodiments described herein. FIG. 4A illustrates in detail memory address circuitry 410A, which may be the same as the memory address circuitry 110A in FIG. 1A. The memory address circuitry 410A differs from the memory address circuitry 310A in that the output of each of the adders 23i is coupled to an input of a linear-feedback shift register (LFSR) 46i, and the output of each LFSR 46i is coupled to the ADDR terminal of the memory 12iIn operation, each of the adders 23i may be configured to add the address received from the ADDR terminal of the receive circuitry 10i to a seed value received from the seed circuitry 240. The seed provided to each adder 23i may be different, and may function as an offset value. Each adder 23i may be configured to provide the sum of the address received from the ADDR terminal of the receive circuitry 10i and the seed value received from the seed circuitry 240 to the LFSR 46i. The LFSR 46i may be configured to generate a pseudorandom value based on the sum of the address received from the ADDR terminal of the receive circuitry 10i and the seed value received from the seed circuitry 240. The memory 12i may be configured to write the word of ultrasound data received at the DIN terminal from the DOUT terminal of the receive circuitry 10i to the memory address that is equivalent to the pseudorandom value generated based on the sum of the address received from the ADDR terminal of the receive circuitry 10i and the seed value received from the seed circuitry 240.

The output of the memory address circuitry 410A may thus be LFSR(address+seed mod N), where address is received from the ADDR terminal of the receive circuitry 10i, seed is the seed received from the seed circuitry 240, and LFSR(n) is a function that generates a pseudorandom value based on n. In particular, in FIG. 4A, for each word of ultrasound data outputted by the receive circuitry 10i, the LFSR may be initialized with (address+seed), and then one iteration cycle of the LFSR may occur. In this cycle, the next value of the LFSR may be computed based on (address+seed) using a particular polynomial, and outputted by the memory address circuitry 410A as the remapped address. The modulus N may occur if the bit width of the address lines can accommodate values from 0 to N−1.

The LFSR may be configured not to output repeated addresses. In particular, the LFSR may be configured with a maximal polynomial that has a period of 2^{N-1}, where N is the number of bits. Given any non-zero starting value, such an LFSR will produce all other values (uniquely) until the LFSR again outputs the starting value. The LFSR will not output 0 during this cycle. Given a starting value of 0, the LFSR will produce 0 for every subsequent iteration. Thus, for input address values ranging from 0 to 2^{N-1}, LFSR(address+seed) may not output a repeated remapped address. Table 4 illustrates examples of addresses, seeds, and remapped addresses, where the remapped address is LFSR(address+seed mod N), and the polynomial is x⁴+x³+1. If (address+seed mod N) is a 4-bit value called [sum(1) sum(2) sum(3) sum(4)], then the remapped address for this polynomial may be [xor(sum(4), sum(3)) sum(1) sum(2) sum(3)]. The case in which a starting value of 0 for (address+seed mod N) is remapped to 0 is a special case, as described above.

TABLE 4
Examples of addresses, seeds, and remapped addresses,
where the remapped address is LFSR(address + seed mod
N) and the polynomial is x⁴+ x³ + 1

| | | | | Remapped | Remapped |
|---|---|---|---|---|---|
| Address | Address | Seed | Seed | Address | Address |
| (Binary) | (Decimal) | (Binary) | (Decimal) | (Binary) | (Decimal) |
| 0111 | 7 | 0001 | 1 | 0100 | 4 |
| 0111 | 7 | 0011 | 3 | 1101 | 13 |
| 1000 | 8 | 0001 | 1 | 1100 | 12 |
| 1000 | 8 | 0011 | 3 | 0101 | 5 |
| 0000 | 0 | 0000 | 0 | 0000 | 0 |

FIG. 4B is a schematic diagram illustrating example circuitry in an ultrasound device, in accordance with certain embodiments described herein. FIG. 4B illustrates in detail memory address circuitry 410B, which may be the same as the memory address circuitry 110B in FIG. 1B. The memory address circuitry 410B differs from the memory address circuitry 410A in that the output of each LFSR 46i is coupled to the ADDR terminal of each memory 12i. The ADDR terminal of each block of receive circuitry 10i is not coupled to the memory address circuitry 410B. The seed circuitry 240 is coupled to each LFSR 46i and is configured to provide the seed for each LFSR 46i. The output of the memory address circuitry 410B may thus be LFSR(seed mod N). In particular, the LFSR may be initialized at the outset with seed, and then for each word of ultrasound data outputted by the receive circuitry 10i, one iteration cycle of the LFSR may occur. In each cycle, the next value of the LFSR may be computed based on the previous value of the LFSR using a particular polynomial, and outputted by the memory address circuitry 410B as the remapped address. The output on a given clock cycle from each LFSR 46i may therefore not depend on an address outputted by receive circuitry 10i, but rather may depend just on the specific seed received from the seed circuitry 240 for a given block of receive circuitry 10i. Table 5 illustrates examples of seeds and remapped addresses, where the remapped address is LFSR(seed mod N), and the polynomial is x⁴+x³+1. If the seed is a 4-bit value called [seed(1) seed(2) seed(3) seed(4)], then the remapped address for this polynomial may be =[xor(seed(4), seed(3)) seed(1) seed(2) seed(3)]. The case in which a starting value of 0 for (seed mod N) is remapped to 0 is a special case, as described above.

TABLE 5
Examples of seeds and remanned addresses, where the remanned address
is LFSR(seed mod N) and the polynomial is x⁴ + x³ + 1

| | | Remapped | Remapped |
|---|---|---|---|
| Seed | Seed | Address | Address |
| (Binary) | (Decimal) | (Binary) | (Decimal) |
| 0001 | 1 | 1000 | 8 |
| 0011 | 3 | 0001 | 1 |
| 0000 | 0 | 0000 | 0 |

The memory address circuitry 410B may be sufficient compared with the memory address circuitry 410A, in which f(address+seed mod N)=LFSR(address+seed mod N), when the receive circuitry 10i outputs memory addresses that simply increase linearly (e.g., increment by 1) on each clock cycle. However, the memory address circuitry 410A may be more appropriate when receive circuitry 10i includes circuitry (e.g., beamforming circuitry) configured to output specific addresses that may not be in succession. In such cases, it may be helpful for the new memory addresses to depend on the address received from the receive circuitry 10i.

The LFSR may be configured not to output repeated addresses. In particular, the LFSR may be configured with a maximal polynomial that has a period of 2^{N-1}, where N is the number of bits. Given any non-zereo starting value, such an LFSR will produce all other values (uniquely) until the LFSR again outputs the starting value. The LFSR will not output 0 during this cycle. Given a starting value of 0, the LFSR will produce 0 for every subsequent iteration. Thus, assuming a non-zero seed, LFSR(seed) may not output a repeated remapped address for 2^{N-1} cycles, during which the LFSR may output remapped addresses from 1 to 2^{N-1}. To cover all addresses between 0 and 2^{N-1}, the circuitry in FIG. 4B may be configured to output address 0 to the memory 12i before, after, or at some time during the LFSR's cycle, and otherwise the LFSR may output a remapped address to the memory 12i.

FIG. 4D is a schematic diagram illustrating example circuitry in an ultrasound device, in accordance with certain embodiments described herein. FIG. 4D illustrates in detail memory address circuitry 410D, which may be the same as the memory address circuitry 110B in FIG. 1B. The memory address circuitry 410D differs from the memory address circuitry 310A in that a single counter 470 is configured to generate a memory address that is the input to the adders 23i, rather than each block of receive circuitry 10i outputting the memory address that is the input to the adders 23i. The output of the memory address circuitry 210 thus may be gray_encoding(address+seed mod N), where address is received from the counter 470, seed is the particular seed (which may function as an offset value) received from the seed circuitry 240 for the receive circuitry 10i, and gray_encoding(n) is a function that transforms a binary-coded value n to a gray-coded value. The modulus N may occur due to the bit widths of the address lines accommodating values from 0 to N−1.

FIG. 4E is a schematic diagram illustrating example circuitry in an ultrasound device, in accordance with certain embodiments described herein. FIG. 4E illustrates in detail memory address circuitry 410C, which may be the same as the memory address circuitry 110B in FIG. 1B. The memory address circuitry 410E differs from the memory address circuitry 410A in that a single counter 470 is configured to generate a memory address that is the input to the adders 23i, rather than each block of receive circuitry 10i outputting the memory address that is the input to the adders 23i. The output of the memory address circuitry 210 thus may be LFSR(address+seed mod N), where address is received from the counter 470, seed is the particular seed (which may function as an offset value) received from the seed circuitry 240 for the receive circuitry 10i, and LFSR(n) is a function that generates a pseudorandom value based on n. In particular, in FIG. 4E, for each word of ultrasound data outputted by the receive circuitry 10i, the LFSR may be initialized with (address+seed), and then one iteration cycle of the LFSR may occur. In this cycle, the next value of the LFSR may be computed based on (address+seed) using a particular polynomial, and outputted by the memory address circuitry 410E as the remapped address. The LFSR may be configured not to output repeated addresses. In particular, the polynomial of the LFSR may have a period (i.e., a number of iterations during which no repeated outputs occur) that is larger than the number of addresses in the memory 12i. The modulus N may occur due to the bit widths of the address lines accommodating values from 0 to N−1.

It should be appreciated that the memory address circuitry 110A, 110B, 210, 310A, 410A, 410B, 410C, 410D, and 410E may be configured to map a single memory address to a different address for each block of receive circuitry 10i, or generate a different address for each block of receive circuitry 10i. In other words, each block of receive circuitry 10i (or at least certain blocks of receive circuitry 10i) may write words of ultrasound data to a different memory address on a given clock cycle. Thus, rather than all the blocks of memory 12i simultaneously undergoing a transition from one memory address to another that may cause a power disturbance (e.g., a transition that includes flipping a large number of bits and/or flipping higher order bits), different blocks of memory 12i may undergo these transitions at different times. This may reduce the total power disturbance caused by such transitions at any given time.

It should be appreciated that the schematic illustrations in FIGS. 1-4E are non-limiting, and there may be more or less circuitry than shown. For example, while two components may be shown as directly coupled together, in some embodiments, there may be other components coupled between. In some embodiments, multiple blocks of receive circuitry 10i may share a single block of memory 12i. In some embodiments, the addresses outputted by certain blocks of receive circuitry 10i may be remapped by memory address circuitry but other addresses may not be remapped. While the above description has described LFSRs for generating pseudorandom values, other types of pseudorandom value generation circuitry may be used. The examples of memory address circuitry illustrated in FIGS. 1-4E are non-limiting, and other types of circuitry for remapping or generating memory addresses using other remapping or generation schemes may also be used.

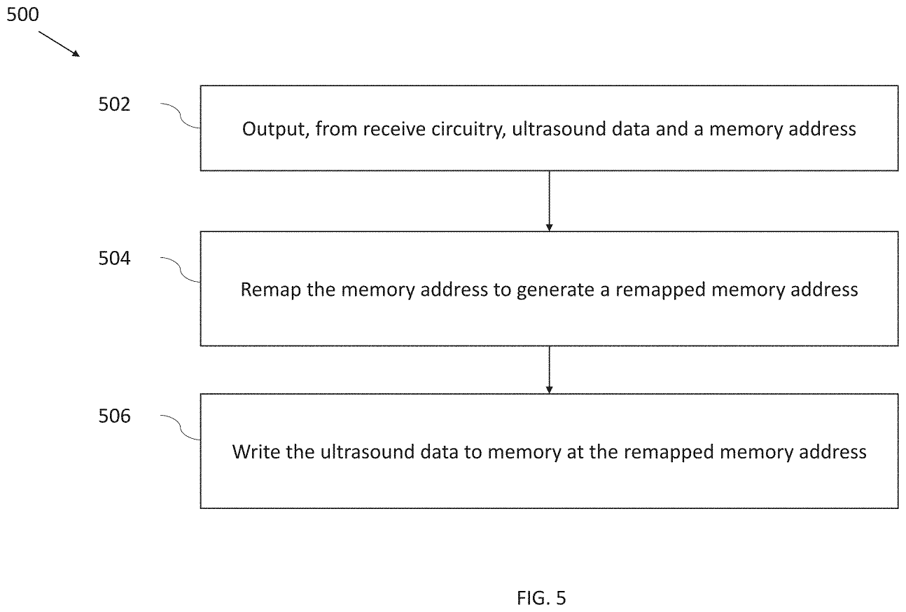

FIG. 5 is a flow diagram illustrating an example process 500 for storing ultrasound data, in accordance with certain embodiments described herein. The process 500 may be performed by an ultrasound device (e.g., ultrasound-on-chip).

The process begins at act 502. In act 502, the ultrasound device outputs, from receive circuitry (e.g., the receive circuitry 101, 102 . . . 10n), ultrasound data and a memory address. The receive circuitry may be configured to generate the ultrasound data (e.g., a word of ultrasound data) by receiving ultrasound signals from one or more ultrasonic transducers and processing them. The receive circuitry may include, for example, amplification circuitry, analog filtering circuitry, analog beamforming circuitry, analog dechirp circuitry, analog quadrature demodulation (AQDM) circuitry, analog time delay circuitry, analog phase shifter circuitry, analog summing circuitry, analog time gain compensation circuitry, analog averaging circuitry, analog-to-digital conversion circuitry, digital filtering, digital beamforming circuitry, digital quadrature demodulation (DQDM) circuitry, digital averaging circuitry, digital dechirp circuitry, digital time delay circuitry, digital phase shifter circuitry, digital summing circuitry, and/or digital multiplying circuitry. The receive circuitry may output the ultrasound data and memory address on a given clock cycle. To generate the memory address, the receive circuitry may include a counter configured to output, at each clock cycle a value that increases linearly from the previous value (e.g., is the value from the previous clock cycle incremented by 1). However, in some embodiments, the receive circuitry may include circuitry (e.g., beamforming circuitry) configured to output specific addresses that may not be in succession. The process 500 proceeds from act 502 to act 504.

In act 504, the ultrasound device remaps (e.g., by memory address circuitry such as the memory address circuitry 110A, 210, 310A, 310B, 410A) the memory address (outputted in act 502) to generate a remapped memory address. Remapping a memory address may include mapping the memory address to a new address using a mapping of the memory address space onto itself. In some embodiments, referring to the memory address received in act 502 as “address,” the remapped memory address may be f(address+seed mod N), where seed is a specific value for the receive circuitry, f is a function, and the available memory addresses range from 0 to N−1. In some embodiments, f(address+seed mod N)=(address+seed mod N). In other words, the remapped memory address may be offset from address by seed. In some embodiments, f may be a function that transforms a memory address from standard binary coding to gray coding. As another example, f may be a function that generates a pseudorandom memory address based on address, which is the remapped memory address. The seed for the block of receive circuitry may be, for example, related to the receive circuitry's physical location (e.g., its location in an ultrasound-on-chip) or a pseudorandom value. In some embodiments, new addresses may be generated based just on an address, not a seed. The process 500 proceeds from act 504 to act 506.

In act 506, the ultrasound device writes the ultrasound data (received in act 502) to memory (i.e., memory 121, 122 . . . 12n, where the specific block of memory corresponds to the receive circuitry of act 502). Writing the ultrasound data at the remapped memory address may include summing the ultrasound data received in act 502 with the existing data at the remapped memory address in the memory (in other words, accumulating) or overwriting the existing data at the remapped memory address in the memory with the ultrasound data received in act 502.

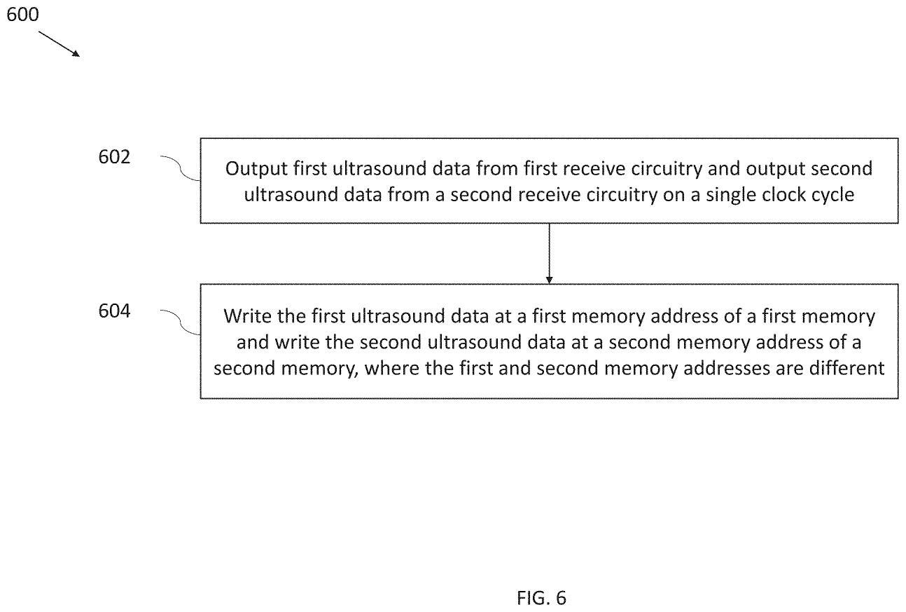

FIG. 6 is a flow diagram illustrating an example process 600 for storing ultrasound data, in accordance with certain embodiments described herein. The process 600 may be performed by an ultrasound device (e.g., an ultrasound-on-chip).

The process begins at act 602. In act 602, the ultrasound device outputs first ultrasound data from first receive circuitry (e.g., the receive circuitry 111). Also in act 602, the ultrasound device outputs second ultrasound data from second receive circuitry (e.g., the receive circuitry 112). The ultrasound device receives the first and second ultrasound data on a single clock cycle. Each of the first and second receive circuitry may be configured to generate the respective ultrasound data by receiving one or more ultrasound signals from one or more ultrasonic transducers and processing them. The first and second receive circuitry may each include, for example, amplification circuitry, analog filtering circuitry, analog beamforming circuitry, analog dechirp circuitry, analog quadrature demodulation (AQDM) circuitry, analog time delay circuitry, analog phase shifter circuitry, analog summing circuitry, analog time gain compensation circuitry, analog averaging circuitry, analog-to-digital conversion circuitry, digital filtering, digital beamforming circuitry, digital quadrature demodulation (DQDM) circuitry, digital averaging circuitry, digital dechirp circuitry, digital time delay circuitry, digital phase shifter circuitry, digital summing circuitry, and/or digital multiplying circuitry. Each of the first and second ultrasound data may be a word of ultrasound data. The process 600 proceeds from act 602 to act 604.

In act 604, the ultrasound device writes the first ultrasound data at a first memory address of a first memory (e.g., the memory 121). Also in act 604, the ultrasound device writes the second ultrasound data at a second memory address of a second memory (e.g., the memory 122). Writing ultrasound data at a memory address may include summing ultrasound data with the existing data at the memory address in other words, accumulating) or overwriting the existing data at the memory address with the new ultrasound data. The first and second memory addresses are different.

In some embodiments, the first and second memory addresses may be results of mapping (e.g., using memory address circuitry 110A, 210, 310A, 310B, or 410A) one memory address (e.g., a single memory address output by both the first and second receive circuitry on the clock cycle) to two different addresses using a mapping of the memory address space onto itself. In some embodiments, the first and second memory address may each be the result of mapping one address to a new address f(address+seed mod N), where seed is different for the first and second receive circuitry, f is a function, and the available memory addresses range from 0 to N−1. In some embodiments, f(address+seed mod N)=(address+seed mod N). In other words, the first and second memory addresses may be linearly offset by different amounts from one memory address. In some embodiments, f may be a function that transforms a memory address from standard binary coding to gray coding. As another example, f may be a function that generates a pseudorandom memory address based on the memory address. The seeds for the first and second receive circuitry may each be, for example, related to the respective receive circuitry's physical location (e.g., its location in an ultrasound-on-chip) or pseudorandom values. In some embodiments, the first and second memory addresses may be results of generating (e.g., using memory address circuitry 110B, 410B, 410C, 410D, or 410E) two different addresses.

As described above, the inventors have recognized that when all the blocks of memory in an ultrasound device store data at one memory address on one clock cycle and then store data at another memory address on the subsequent clock cycle, in some cases the digital switching activity across all the blocks of memory in switching between certain addresses may cause a draw in current from the power supply, power supply noise, and/or transfer of digital switching activity through capacitive coupling to nearby low bandwidth and/or low amplitude analog signals, which can in turn cause noise in images and measurements generates based on the analog signals. The inventors have recognized that such power disturbances may be reduced by implementing remapping of memory addresses, which may include mapping the memory address to a new address using a mapping of the memory address space onto itself. If blocks of receive circuitry output a memory address for storing ultrasound data on a given clock cycle, the memory address circuitry may be configured to map that memory address to new memory addresses (as described with reference to the process 500), a different address for each block of receive circuitry. The inventors have also recognized that such power disturbances may be reduced by implementing generation of a different memory address for each block of receive circuitry (without mapping). Thus, each block of receive circuitry (or at least certain blocks of receive circuitry) may write ultrasound data to a different memory address on a given clock cycle (as described with reference to the process 600). Accordingly, rather than all the blocks of memory simultaneously undergoing a transition from one memory address to another that causes a power disturbance (e.g., a transition that includes flipping a large number of bits and/or flipping higher order bits), different blocks of memory may undergo these transitions at different times. This may reduce the total power disturbance caused by such transitions at any given time.

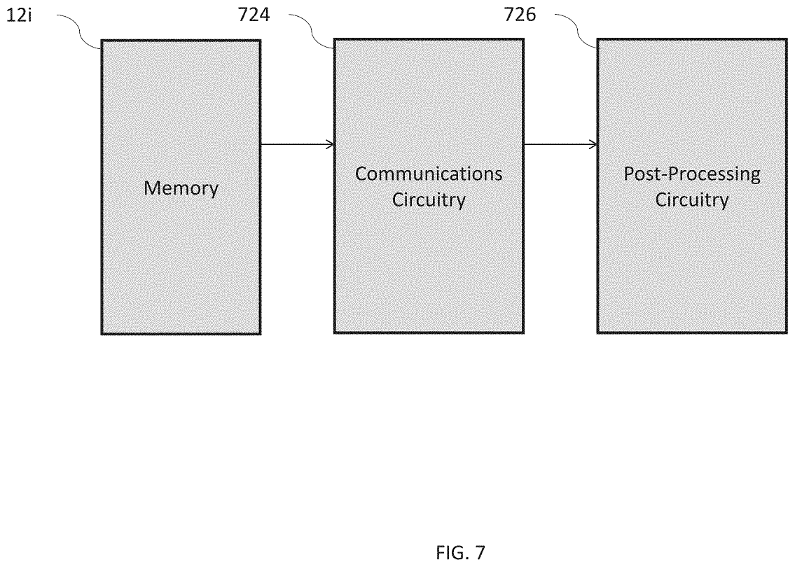

FIG. 7 is a block diagram illustrating an example of a downstream portion of the circuitry in FIGS. 1-4E, in accordance with certain embodiments described herein. FIG. 7 includes memory 12i (i.e., any of the memory 121, 122 . . . 12n), communications circuitry 724, and post-processing circuitry 726. The output terminal of the memory 12i is coupled to the input terminal of communications circuitry 724. The output terminal of the communications circuitry 724 is coupled to the input terminal of post-processing circuitry 726.

The communications circuitry 724 may be configured to transmit data from the memory 12i to the post-processing circuitry 726 and may include, for example, circuitry capable of transmitting data over a communications link such as a Universal Serial Bus (USB) communications link, a serial-deserializer (SerDes) link, or a wireless link (e.g., a link employing the I6 802.11 standard). Thus, the communications circuitry 726 may be coupled to the post-processing circuitry 726 through a USB communications link (e.g., a cable) or through a SerDes communications link. The post-processing circuitry 726 may be configured to post-process ultrasound data after it has been stored in the memory 12i, and may include, for example, circuitry for summing, requantization, noise shaping, waveform removal, image formation, and backend processing. In some embodiments, the memory 12i and the communications circuitry 724 may be located on an ultrasound-on-chip while the post-processing circuitry 726 may be located on a separate electronic device (e.g., a field-programmable gate array (FPGA) device) to which the ultrasound-on chip is coupled. In some embodiments, the memory 12i and the communications circuitry 724 may be located on an ultrasound probe while the post-processing circuitry 726 may be located on a host device to which the ultrasound probe is coupled. In some embodiments, there may be one block of communications circuitry 724 and/or post-processing circuitry 726 per block of memory 12i, while in other embodiments, one block of communications circuitry 724 and/or post-processing circuitry 726 may be shared among multiple blocks of memory 12i.

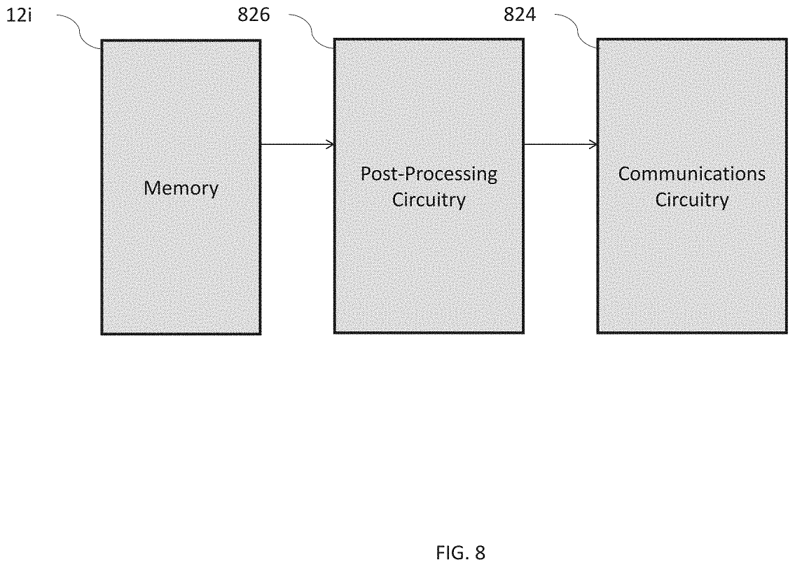

FIG. 8 is a block diagram illustrating another example of a downstream portion of the circuitry in FIGS. 1-4E, in accordance with certain embodiments described herein. FIG. 8 includes the memory 12i (i.e., any of the memory 121, 122 . . . 12n), communications circuitry 824, and post-processing circuitry 826. The DOUT terminal of the memory 12i is coupled to the input terminal of post-processing circuitry 826. The output terminal of the post-processing circuitry 826 is coupled to the input terminal of the communications circuitry 824. The communications circuitry 824 may be configured to transmit data from the post-processing circuitry 826 to another electronic device, such as a host device or an FPGA, and may include, for example, circuitry capable of transmitting data over a communications link such as a Universal Serial Bus (USB) communications link, a serial-deserializer (SerDes) link, or a wireless link (e.g., a link employing the I6 802.11 standard). The post-processing circuitry 826 may be configured to post-process ultrasound data after it has been stored in the memory 12i, and may include, for example, circuitry for summing, requantization, noise shaping, waveform removal, image formation, and backend processing. In some embodiments, the memory 12i, the post-processing circuitry 826, and the communications circuitry 824 may be located on an ultrasound-on-chip. In some embodiments, the memory 12i, the post-processing circuitry 826, and the communications circuitry 824 may be located on an ultrasound probe. In some embodiments, there may be one block of communications circuitry 824 and/or post-processing circuitry 826 per block of memory 12i, while in other embodiments, one block of communications circuitry 824 and/or post-processing circuitry 826 may be shared among multiple blocks of memory 12i.

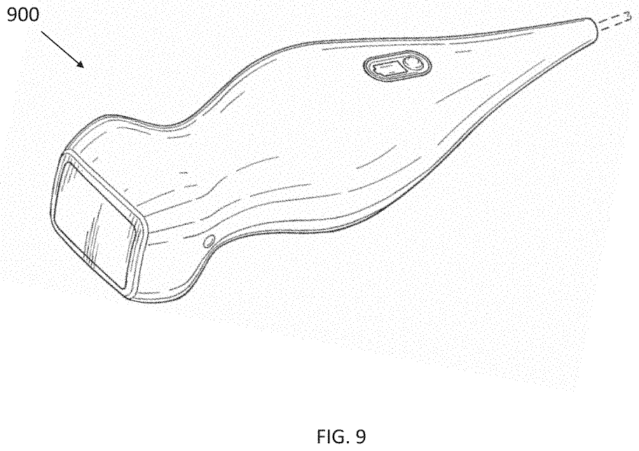

FIG. 9 is a perspective view of an example handheld ultrasound probe 900 in which an ultrasound-on-device may be disposed, in accordance with certain embodiments described herein. The ultrasound-on-chip in the handheld ultrasound probe 900 may include all of the receive circuitry described herein.

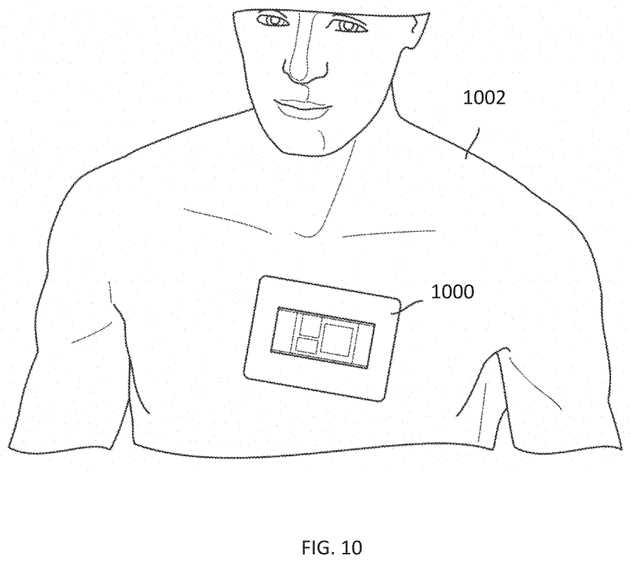

FIG. 10 illustrates a subject 1002 wearing an example ultrasound patch 1000 in which an ultrasound-on-device may be disposed, in accordance with certain embodiments described herein. The ultrasound patch 1000 is coupled to the subject 1002. The ultrasound-on-chip in the ultrasound patch 800 may include all of the receive circuitry described herein.

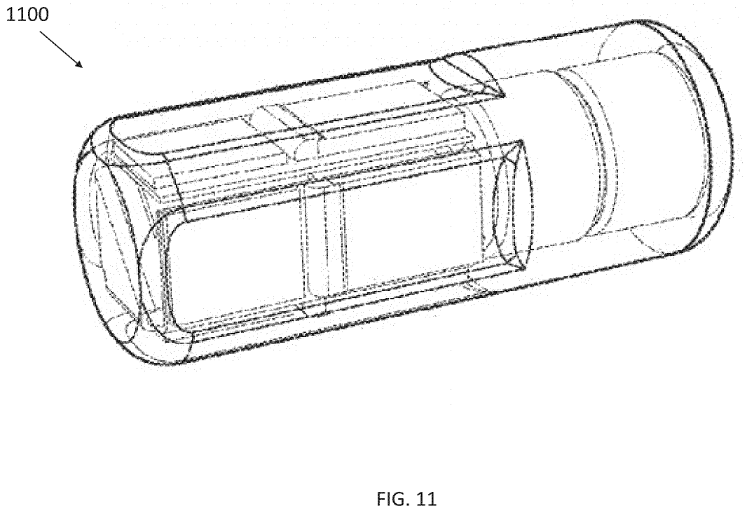

FIG. 11 is a perspective view of an example ultrasound pill 1100 in which an ultrasound-on-device may be disposed, in accordance with certain embodiments described herein. The ultrasound-on-chip in the ultrasound patch 1100 may include all of the receive circuitry described herein.

Further description of the handheld ultrasound probe 900, the ultrasound patch 1000, and the ultrasound pill 1100 may be found in U.S. patent application Ser. No. 15/826,711 titled “UNIVERSAL ULTRASOUND IMAGING DEVICE AND RELATED APPARATUS AND METHODS,” filed on Jun. 19, 2017 and published as U.S. Pat. App. Publication No. 2017-0360399 A1 (and assigned to the assignee of the instant application).

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound apparatus comprising:
first receive circuitry;
second receive circuitry;
first memory; and
second memory;
wherein the ultrasound apparatus is configured to:
output first ultrasound data from the first receive circuitry and output second ultrasound data from the second receive circuitry on a single clock cycle; and
write the first ultrasound data at a first memory address of the first memory and write the second ultrasound data at a second memory address of the second memory, wherein the first and second memory addresses are different.

2. The ultrasound apparatus of claim 1, further comprising memory address circuitry configured to generate the first memory address and the second memory address.

3. The ultrasound apparatus of claim 2, wherein the memory address circuitry is configured to remap a memory address received from the first receive circuitry to generate the first memory address and to remap a memory address received from the second receive circuitry to generate the second memory address.

4. The ultrasound apparatus of claim 2, wherein:
the memory address circuitry is configured to:
add a memory address received from the first receive circuitry to a first seed value in order to generate the first memory address; and
add a memory address received from the second receive circuitry to a second seed value in order to generate the second memory address; and
the first seed value and the second seed value are different.

5. The ultrasound apparatus of claim 2, wherein:
the memory address circuitry is configured to:
add a memory address received from the first receive circuitry to a first seed value in order to generate a first sum;
add a memory address received from the second receive circuitry to a second seed value in order to generate a second sum;
gray encode the first sum in order to generate the first memory address; and
gray encode the second sum in order to generate the second memory address; and
the first seed value and the second seed value are different.

6. The ultrasound apparatus of claim 2, wherein:
the memory address circuitry is configured to:
add a memory address received from the first receive circuitry to a first seed value in order to generate a first sum;
add a memory address received from the second receive circuitry to a second seed value in order to generate a second sum;
generate a first pseudorandom value based on the first sum, wherein the first pseudorandom value is the first memory address; and
generate a second pseudorandom value based on the second sum, wherein the second pseudorandom value is the first memory address; and
the first seed value and the second seed value are different.

7. The ultrasound apparatus of claim 2, wherein:
the memory address circuitry is configured to:
generate a counter value on each clock cycle;
add the counter value to a first seed value in order to generate the first memory address;
add the counter value to a second seed value in order to generate the second memory address; and
the first seed value and the second seed value are different.

8. The ultrasound apparatus of claim 2, wherein:
the memory address circuitry is configured to:
generate a counter value on each clock cycle;
add the counter value to a first seed value in order to generate a first sum;
add the counter value to a second seed value in order to generate a second sum;
gray encode the first sum in order to generate the first memory address; and
gray encode the second sum in order to generate the second memory address; and
the first seed value and the second seed value are different.

9. The ultrasound apparatus of claim 2, wherein:
the memory address circuitry is configured to:
generate a counter value on each clock cycle;
add the counter value to a first seed value in order to generate a first sum;
add the counter value to a second seed value in order to generate a second sum;
generate a first pseudorandom value based on the first sum, wherein the first pseudorandom value is the first memory address; and
generate a second pseudorandom value based on the second sum, wherein the second pseudorandom value is the first memory address; and
the first seed value and the second seed value are different.

10. The ultrasound apparatus of claim 2, wherein:
the memory address is configured to:
generate a first pseudorandom value based on a first seed value, wherein the first pseudorandom value is the first memory address; and
generate a second pseudorandom value based on a second seed value, wherein the second pseudorandom value is the second memory address; and
the first seed value and the second seed values are different.

11. The ultrasound apparatus of claim 10, further comprising pseudorandom value generation circuitry configured to generate the first and second pseudorandom values.

12. The ultrasound apparatus of claim 11, wherein the pseudorandom value generation circuitry comprises a linear-feedback shift register (LFSR).

13. The ultrasound apparatus of claim 12, further comprising storage circuitry for storing the first and second seed values.

14. The ultrasound apparatus of claim 13, wherein the first seed value is related to a location of the first receive circuitry and the second seed value is related to a location of the second receive circuitry.

15. The ultrasound apparatus of claim 14, wherein the location of the first receive circuitry and the location of the second receive circuitry are locations in an ultrasound-on-chip.

16. The ultrasound apparatus of claim 12, further comprising pseudorandom value generation circuitry for generating the first and second seed values.

17. The ultrasound apparatus of claim 16, wherein the pseudorandom value generation circuitry comprises a linear-feedback shift register (LFSR).

18. The ultrasound apparatus of claim 17, wherein the memory address received from the first receive circuitry and the memory address received from the second receive circuitry are the same.

19. The ultrasound apparatus of claim 18, wherein:
the first receive circuitry comprises a first counter, and the address received from the first receive circuitry is generated by the first counter;
the second receive circuitry comprises a second counter, and the address received from the second receive circuitry is generated by the second counter.

20. The ultrasound apparatus of claim 18, wherein:
the first receive circuitry comprises first circuitry configured to generate addresses not in succession, and the address received from the first receive circuitry is generated by the first circuitry;
the second receive circuitry comprises second circuitry configured to generate addresses not in succession, and the address received from the second receive circuitry is generated by the second circuitry.

21. The ultrasound apparatus of claim 20, wherein the first and second circuitry comprises beamforming circuitry.

22. The ultrasound apparatus of claim 1, wherein the ultrasound apparatus is configured, when writing the first ultrasound data at the first memory address of the first memory and writing the second ultrasound data at the second memory address of the second memory, to:
sum the first ultrasound data with existing data at the first memory address of the first memory; and
sum the second ultrasound data with existing data at the second memory address of the second memory.

23. The ultrasound apparatus of claim 1, wherein the ultrasound apparatus is configured, when writing the first ultrasound data at the first memory address of the first memory and writing the second ultrasound data at the second memory address of the second memory, to:
overwrite existing data at the first memory address of the first memory with the first ultrasound data; and
overwrite existing data at the second memory address of the second memory with the second ultrasound data

24. The ultrasound apparatus of claim 1, wherein the first and second receive circuitry each comprise amplification circuitry, analog filtering circuitry, analog beamforming circuitry, analog dechirp circuitry, analog quadrature demodulation (AQDM) circuitry, analog time delay circuitry, analog phase shifter circuitry, analog summing circuitry, analog time gain compensation circuitry, analog averaging circuitry, analog-to-digital conversion circuitry, digital filtering, digital beamforming circuitry, digital quadrature demodulation (DQDM) circuitry, digital averaging circuitry, digital dechirp circuitry, digital time delay circuitry, digital phase shifter circuitry, digital summing circuitry, and/or digital multiplying circuitry.
